(19) United States
(12) Patent Application Publication
(10) Pub. No.: US 2022/0015717 A1
(43) Pub. Date: Jan. 20, 2022

(54) Activity State Analysis Device, Activity State Analysis Method and Activity State Analysis System

(71) Applicant: Nippon Telegraph and Telephone Corporation, Tokyo (JP)

(72) Inventors: Takayuki Ogasawara, Tokyo (JP); Rieko Sato, Tokyo (JP); Masahiko Mukaino, Tokyo (JP); Hirotaka Matsuura, Tokyo (JP); Eiichi Saitoh, Tokyo (JP)

(21) Appl. No.: 17/298,842

(22) PCT Filed: Nov. 28, 2019
(86) PCT No.: PCT/JP2019/046614

§ 371 Date: Jun. 1, 2021

(30) Foreign Application Priority Data
Dec. 12, 2018 (JP) 2018-232164

(51) Int. Cl. A61B 5/00; A61B 5/0255; A61B 5/11; A61B 5/0245; G16H 50/30; G16H 40/67

(52) U.S. Cl. CPC A61B 5/7278; A61B 5/0255; A61B 5/1118; G16H 40/67; A61B 5/0245; G16H 50/30; A61B 5/4806

(57) ABSTRACT

An embodiment activity state analysis device includes a measurement unit configured to measure a piece of biological information including a heart rate or a pulse rate of a user, a storage unit configured to store a plurality of the pieces of biological information measured in time series, a representative value calculation unit configured to acquire the plurality of the pieces of biological information measured when the user is resting from the storage unit and calculate a representative value, and an activity state calculation unit configured to calculate an activity state of the user by using the representative value calculated by the representative value calculation unit.

This patent application is a national phase filing under section 371 of PCT/JP2019/046614, filed Nov. 28, 2019, which claims the priority of Japanese patent application no. 2018-232164, filed Dec. 12, 2018, each of which is incorporated herein by reference in its entirety.

### TECHNICAL FIELD

The present disclosure relates to an activity state analysis device, an activity state analysis method, and an activity state analysis system, and particularly to a technique for analyzing biological information measured by a sensor.

### BACKGROUND

In recent years, indicators such as motion intensities and physiological cost indexes have been used as means for ascertaining features of the body in sports and rehabilitation medical services. Activity states such as motionless and walking states of the bodies of athletes, rehabilitation patients, and the like are analyzed using motion intensities and physiological cost indexes, thereby enabling safer and more effective training and rehabilitation.

Motion intensities and physiological cost indexes are calculated using the following equations (see Non Patent Literatures 1 and 2).

Equation (1)

(Motion intensity)=(heart rate−resting heart rate)/(maximum heart rate−resting heart rate)×100 (1)

Equation (2)

(Physiological cost index)=(heart rate while walking−resting heart rate)/(walking speed) (2)

The “resting heart rate” used in Equations (1) and (2) described above is biological information serving as a reference value at the time of obtaining a motion intensity and a physiological cost index.

For example, Non Patent Literature 3 discloses a measurement method in which data of one heart rate measured by a user through his or her own operation when the user is awake in the morning and in a lying state is set as a resting heart rate.

### CITATION LIST

### Non Patent Literature

Non Patent Literature 1: “Motion intensity,” [online], Dec. 9, 2017, Wikipedia, [retrieved Jun. 12, 2018],

Internet<https://ja.wikipedia.org/wiki/

[symbol]

[symbol]

Non Patent Literature 2: “Physiological cost index (PCI): Clinical indicator of physiological cost index,” [online], Feb. 11, 2015, [retrieved Jun. 12, 2018], Internet

http://rigakuryouhourinshou.blog.fc2.com/blog-entry-158.html?sp.

Non Patent Literature 3: “Learn motion intensity from maximum heart rate and resting heart rate,” [online], Jun. 9, 2017, [retrieved Jun. 12, 2018], Internet

https://infomation.club/training-general/know-intencity/#i-2.

Non Patent Literature 4: Niijima, A Study of Sleep-Wake Detection Algorithm Based on Activity Counts and Idleness Periods with an Accelerometer, IEICE Technical Report 115 (486), 1-6, 2016-03-03, Institute of Electronics, Information and Communication Engineers.

Non Patent Literature 5: Miyamoto, “Wearable Sensors Corresponding to Various Applications in Healthcare Field,” Toshiba Review Vol. 69 No. 11, 13-16 (2014).

Non Patent Literature 6: Kasai, “Measurement of Nocturnal Sleep State by a Mattress-type Sleep Monitor in a Nursing Home,” Japanese Journal of Nursing Art and Science Vol. 14, No. 2, pp. 195-199, 2015.

### SUMMARY

### Technical Problem

However, in the related art, even in a state where a user is lying face down, one heart rate measured at the time when the user's body moves is set as a resting heart rate, thereby making it difficult to obtain the value of a stable resting heart rate. For this reason, it is difficult to improve the reliability of an indicator of an activity state calculated on the basis of a resting heart rate.

Embodiments of the present disclosure are contrived to solve the above-described problems, and an object thereof is to provide an activity state analysis device, an activity state analysis method, and an activity state analysis system which are capable of obtaining a highly reliable indicator indicating an activity state of a user on the basis of the value of a stable resting heart rate.

### Means for Solving the Problem

In order to solve the problems described above, an activity state analysis device according to embodiments of the present disclosure includes a measurement unit configured to measure a piece of biological information including a heart rate or a pulse rate of a user, a storage unit configured to store a plurality of the pieces of biological information measured in time series, a representative value calculation unit configured to acquire, from the storage unit, the plurality of the pieces of biological information measured when the user is resting and calculate a representative value, and an activity state calculation unit configured to calculate an activity state of the user by using the representative value calculated by the representative value calculation unit.

Further, in the activity state analysis device according to embodiments of the present disclosure, the representative value calculation unit may acquire, from the storage unit, the plurality of the pieces of biological information measured in a set time period, the plurality of the pieces of biological information measured at a plurality of points in time when the user is estimated to be in a lying position from an acceleration of the user, or the plurality of the pieces of biological information measured at a plurality of points in time when the user is estimated to be in a sleeping state, as the plurality of pieces of biological information measured when the user is resting.

In order to solve the problems described above, an activity state analysis method according to embodiments of the present disclosure includes a measurement step of measuring a piece of biological information including a heart rate or a pulse rate of a user, a storage step of storing a plurality of the pieces of biological information measured in time series in a storage unit, a representative value calculation step of acquiring, from the storage unit, the plurality of the pieces of biological information measured when the user is resting and calculating a representative value, and an activity state calculation step of calculating an activity state of the user by using the representative value calculated in the representative value calculation step.

Further, in the activity state analysis method according to embodiments of the present disclosure, the representative value calculation step may include acquiring, from the storage unit, the plurality of the pieces of biological information measured in a set time period, the plurality of the pieces of biological information measured at a plurality of points in time when the user is estimated to be in a lying position from an acceleration of the user, or the plurality of the pieces of biological information measured at a plurality of points in time when the user is estimated to be in a sleeping state, as the plurality of pieces of biological information measured when the user is resting.

In order to solve the problems described above, an activity state analysis system according to embodiments of the present disclosure includes a sensor terminal configured to output, to an outside, a piece of biological information including a heart rate or a pulse rate of a user which are measured by a sensor, a relay terminal configured to receive the piece of biological information output from the sensor terminal and output the piece of biological information to an outside, and an external terminal configured to receive the piece of biological information output from the sensor terminal or the relay terminal and display the piece of biological information on a display device, in which at least one of the sensor terminal, the relay terminal, or the external terminal includes a storage unit configured to store a plurality of the pieces of biological information measured in time series, a representative value calculation unit configured to acquire, from the storage unit, the plurality of the pieces of biological information measured when the user is resting and calculate a representative value, and an activity state calculation unit that calculates an activity state of the user using the representative value calculated by the representative value calculation unit.

In order to solve the problems described above, an activity state analysis system according to embodiments of the present disclosure includes a sensor terminal configured to include a first analysis unit, a relay terminal configured to include a second analysis unit, and an external terminal configured to include a third analysis unit, in which the sensor terminal outputs, to an outside, biological information including a heart rate or a pulse rate of a user measured by a sensor, the relay terminal receives the piece of biological information of the user output from the sensor terminal and outputs the piece of biological information to an outside, the external terminal receives the piece of biological information output from the sensor terminal or the relay terminal and displays the piece of biological information on a display device, at least one of the sensor terminal, the relay terminal, or the external terminal includes a storage unit configured to store a plurality of the pieces of biological information measured in time series, and the first analysis unit, the second analysis unit, and the third analysis unit cause, in cooperation with each other, a representative value calculation unit and an activity state calculation unit to be implemented, the representative value calculation unit acquiring, from the storage unit, the plurality of pieces of biological information measured when the user is resting and calculating a representative value, and the activity state calculation unit calculating an activity state of the user by using the representative value calculated by the representative value calculation unit.

Further, in the activity state analysis system according to embodiments of the present disclosure, the representative value calculation unit may acquire, from the storage unit, the plurality of the pieces of biological information measured in a set time period, the plurality of the pieces of biological information measured at a plurality of points in time when the user is estimated to be in a lying position from an acceleration of the user, or the plurality of the pieces of biological information measured at a plurality of points in time when the user is estimated to be in a sleeping state, as the plurality of pieces of biological information measured when the user is resting.

Further, in the activity state analysis system according to embodiments of the present disclosure, the representative value may be a median value or an average value.

### Effects of Embodiments of the Invention

According to embodiments of the present disclosure, a representative value of a plurality of heart rate measurements of a user which are measured in a resting state of the user is calculated as a resting heart rate, and thus it is possible to obtain a highly reliable indicator indicating an activity state of the user on the basis of the value of a stable resting heart rate.

### BRIEF DESCRIPTION OF THE DRAWINGS

FIG. 1 is a block diagram illustrating functions of an activity state analysis device according to a first embodiment of the present disclosure.

FIG. 2 is a block diagram illustrating a hardware configuration of the activity state analysis device according to the first embodiment.

FIG. 3 is a flowchart illustrating operations of the activity state analysis device according to the first embodiment.

FIG. 4 is a diagram illustrating a configuration of an activity state analysis system according to the first embodiment.

FIG. 5 is a block diagram illustrating a configuration of the activity state analysis system according to the first embodiment.

FIG. 6 is a sequence diagram illustrating operations of the activity state analysis system according to the first embodiment.

FIG. 7 is a diagram illustrating effects of the activity state analysis device according to the first embodiment.

FIG. 8 is a diagram illustrating effects of the activity state analysis device according to the first embodiment.

FIG. 9 is a block diagram illustrating a configuration of an activity state analysis device according to a second embodiment.

FIG. 10 is a block diagram illustrating a configuration of a lying position estimation unit according to the second embodiment.

FIG. 11 is a sequence diagram illustrating operations of an activity state analysis system according to the second embodiment.

FIG. 12 is a sequence diagram illustrating operations of an activity state analysis system according to a third embodiment.

FIG. 13 is a block diagram illustrating a configuration of an activity state analysis device according to a fourth embodiment.

FIG. 14 is a sequence diagram illustrating operations of an activity state analysis system according to the fourth embodiment.

### DETAILED DESCRIPTION OF ILLUSTRATIVE EMBODIMENTS

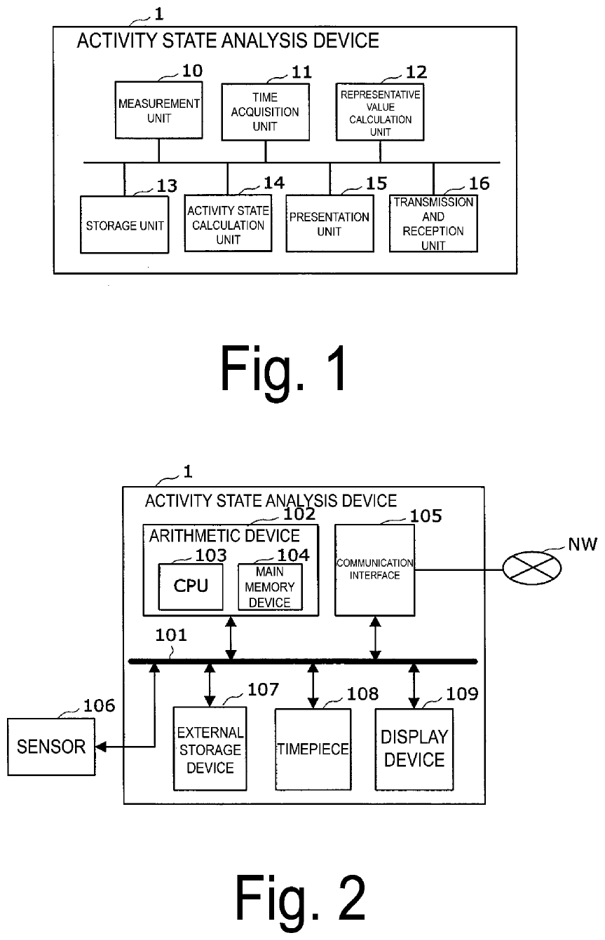

Hereinafter, preferred embodiments of the present disclosure will be described in detail with reference to FIGS. 1 to 14.

### First Embodiment

First, an outline of a configuration of an activity state analysis device 1 according to a first embodiment of the present disclosure will be described. FIG. 1 is a block diagram illustrating a functional configuration of the activity state analysis device 1. The activity state analysis device 1 calculates a representative value of time-series data of a user's heart rate measured in a set time period as a resting heart rate. In addition, the activity state analysis device 1 calculates an indicator indicating an activity state of the user using the calculated resting heart rate, and displays the indicator on a display device or the like as an analysis result. Further, a case where a heart rate is adopted as an example of biological information used for analysis by the activity state analysis device 1 will be described below.

### Functional Blocks of Activity State Analysis Device

The activity state analysis device 1 includes a measurement unit 10, a time acquisition unit 11, a representative value calculation unit 12, a storage unit 13, an activity state calculation unit 14, a presentation unit 15, and a transmission and reception unit 16.

The measurement unit 10 measures a heart rate from an electrocardiographic waveform based on a cardiac potential of a user which is detected by a sensor 106 worn by the user described below. The sensor 106 includes a heart rate meter and the like. The measurement unit 10 measures the user's heart rate, for example, at a sampling rate of one second. The measurement unit 10 outputs time-series data in which a heart rate and a measurement time of digital data are associated with each other. The time-series data of the user's heart rate measured by the measurement unit 10 is stored in the storage unit 13 to be described below. Note that the measurement unit 10 may measure the pulse rate from the pulsation of the user detected by the sensor 106 constituted by, for example, a pulsimeter, instead of a heart rate.

The time acquisition unit 11 acquires time information to be used in the activity state analysis device 1. The time acquisition unit 11 may acquire time information such as a standard time from, for example, a built-in timepiece provided in the activity state analysis device 1 or an NTP server. The time information acquired by the time acquisition unit 11 is used when sampling of a heart rate measured by the measurement unit 10 is performed or when the representative value calculation unit 12 acquires time-series data of the user's heart rate in order to calculate a representative value.

The representative value calculation unit 12 acquires a plurality of heart rate measurements measured in a resting state of the user from the storage unit 13 to calculate a representative value. In more detail, the representative value calculation unit 12 acquires time-series data of the heart rate measured in a set time period from the storage unit 13 to calculate a median value thereof. For example, the representative value calculation unit 12 reads the user's heart rate measured in a time period from 0 a.m. to 5 a.m. from the storage unit 13 as the heart rate measured in a resting state of the user. Note that the representative value calculation unit 12 may acquire a plurality of pulse rate measurements measured in a resting state of the user from the storage unit 13 and calculate a representative value in the same manner.

In addition, the representative value calculation unit 12 may acquire data of the heart rate for five hours from 0 a.m. to 5 a.m. from the storage unit 13 after 5 a.m. with reference to, for example, the current time acquired by the time acquisition unit ii. The median value calculated by the representative value calculation unit 12 is stored in the storage unit 13 as a resting heart rate. Note that the representative value calculation unit 12 may calculate an average value as a representative value of a heart rate.

Here, a time period from 0 a.m. to 5 a.m. of the day is generally a time in which a user such as a patient is sleeping, and thus the user is considered to be in a position that does not involve any proactive activity. From this, the representative value calculation unit 12 obtains a representative value of time-series data of the heart rate in the time period from 0 a.m. to 5 a.m. on the assumption that the user is in a resting state during that time period. In addition, the user's heart rate is measured by configuring a sampling rate to one second in the measurement unit 10, and thus the number of pieces of data of the heart rate for the period of 5 hours is 18000 points. For this reason, it can be said that a median value of the heart rate for the period from 0 a.m. to 5 a.m. is highly reliable.

The storage unit 13 stores time-series data of the user's heart rate measured by the measurement unit 10. The storage unit 13 stores information indicating a “set time period” used by the representative value calculation unit 12, that is, the period from 0 a.m. to 5 a.m. In addition, the storage unit 13 stores a median value of the heart rate calculated by the representative value calculation unit 12 as a resting heart rate. Further, the storage unit 13 previously stores the above-described Equations (1) and (2) that are used when the activity state calculation unit 14 calculates an indicator indicating an activity state.

The activity state calculation unit 14 calculates a motion intensity and a physiological cost index on the basis of the above-described Equations (1) and (2) by using a median value of the heart rate calculated by the representative value calculation unit 12 as a resting heart rate.

The motion intensity and the physiological cost index calculated by the activity state calculation unit 14 are output as analysis results indicating the user's activity state. Note that, in a case where the representative value calculation unit 12 calculates a median value of a pulse rate, the activity state calculation unit 14 uses the median value of the pulse rate as a resting heart rate.

The presentation unit 15 presents analysis results of the user's activity state obtained by the activity state calculation unit 14. In more detail, the presentation unit 15 displays the analysis results on a display device 109 to be described below and generates and presents information for supporting the user on the basis of the analysis results. The presentation unit 15 may output the information for supporting the user to an operation device (not illustrated) realized by the display device 109, a sound output device, a light source, an actuator, heating equipment, or the like.

The transmission and reception unit 16 receives a cardiac potential of the user which is detected by the sensor 106 to be described below. Further, the transmission and reception unit 16 can transmit the analysis results of the user's activity state obtained by the activity state calculation unit 14 to the outside through a communication network.

### Hardware Configuration of Activity State Analysis Device

Next, an example of a hardware configuration of the activity state analysis device 1 having the above-described functions will be described using the block diagram of FIG. 2.

As illustrated in FIG. 2, the activity state analysis device 1 can be implemented by, for example, a computer including an arithmetic device 102 including a CPU 103 and a main memory device 104, a communication interface 105, the sensor 106, an external storage device 107, a timepiece 108, and the display device 109 which are connected to each other through a bus 101, and a program for controlling these hardware resources.

The CPU 103 and the main memory device 104 constitute the arithmetic device 102. A program for the CPU 103 to perform various controls and arithmetic operations is stored in advance in the main memory device 104. The functions of the activity state analysis device 1 including the representative value calculation unit 12 and the activity state calculation unit 14 illustrated in FIG. 1 are implemented by the arithmetic device 102.

The communication interface 105 is an interface and a control device for connecting the activity state analysis device 1 and various external electronic devices through a communication network NW. The activity state analysis device 1 may receive through the communication interface 105 data of biological information such as a cardiac potential, an electrocardiographic waveform, and an acceleration through the communication network NW from the sensor 106 worn by the user described below.

For example, an arithmetic interface and an antenna corresponding to wireless data communication standards such as LTE, 3G, a wireless LAN, or Bluetooth (registered trademark) are used as the communication interface 105. The transmission and reception unit 16 illustrated in FIG. 1 is implemented by the communication interface 105.

The sensor 106 is realized by a sensor such as an electrocardiograph. The sensor 106, which is worn by the user over a preset measurement period, detects and measures biological information such as a cardiac potential of the user.

The external storage device 107 includes a readable and writable storage medium, and a driving device for reading and writing various information, such as programs and data, from and in the storage medium. A hard disk or a semiconductor memory such as a flash memory can be used as a storage medium for the external storage device 107.

The external storage device 107 may include a storage region, a program storage unit, other storage devices not illustrated in the drawings, and the like. Here, the storage region stores an electrocardiographic waveform based on the cardiac potential detected by the sensor 106. In addition, the program storage unit stores a program for the activity state analysis device 1 to perform analysis processing of the activity state of the user. In addition, the storage device is a storage device for backing up, for example, programs, data, and the like stored in the external storage device 107. The storage unit 13 illustrated in FIG. 1 is realized by the external storage device 107.

The timepiece 108, which is constituted by a real-time clock (RTC) including a built-in crystal oscillator provided in the activity state analysis device i, measures the time. Time information obtained by the timepiece 108 is used when the measurement unit 10 performs sampling of a heart rate and the representative value calculation unit 12 acquires a heart rate for calculating a representative value. Note that the time information obtained by the timepiece 108 is acquired by the time acquisition unit 11 illustrated in FIG. 1.

The display device 109 functions as the presentation unit 15 of the activity state analysis device i. The display device 109 is implemented by a liquid crystal display or the like. In addition, the display device 109 constitutes an operation device that outputs user support information generated on the basis of activity state analysis results.

### Operation of Activity State Analysis Device

Next, operations of the activity state analysis device i having the above-described configuration will be described using the flowchart of FIG. 3. In the following description, information indicating a period from 0 a.m. to 5 a.m. is stored in the storage unit 13 as a set condition. First, the following process is executed in a state where the sensor 106 is worn by the user.

The measurement unit 10 measures the user's heart rate from an electrocardiographic waveform based on a cardiac potential detected by the sensor 106 worn by the user (step S1). In more detail, the measurement unit 10 removes noise of the electrocardiographic waveform of the user and calculates a heart rate which is digital data. In addition, the measurement unit 10 performs sampling of the heart rate at a sampling rate of one second.

Next, time-series data of the user's heart rate measured by the measurement unit 10 is stored in the storage unit 13 (step S2). Thereafter, the representative value calculation unit 12 acquires the time-series data of the heart rate measured in a preset time period, that is, the time period from 0 a.m. to 5 a.m., from the storage unit 13 (step S3).

For example, the representative value calculation unit 12 may acquire the heart rate for five fours from 0 a.m. to 5 a.m. from the storage unit 13 after 5 a.m. with reference to the current time acquired by the time acquisition unit 11. Thereafter, the representative value calculation unit 12 calculates a median value of the user's heart rate from 0 a.m. to 5 a.m. acquired in step S3 (step S4). The calculated median value of the heart rate is stored in the storage unit 13 as a resting heart rate of the user.

Next, the activity state calculation unit 14 calculates a motion intensity on the basis of the above-described Equation (1) stored in the storage unit 13, for example, using the user's resting heart rate calculated in step S4 (step S5). The calculated motion intensity is stored in the storage unit 13 as an analysis result of the user's activity state. Note that the activity state calculation unit 14 may similarly calculate a physiological cost index of the user using Equation (2) described above.

Next, the presentation unit 15 displays information indicating the user's motion intensity calculated by the activity state calculation unit 14 on the display device 109 as an analysis result of the user's activity state (step S6). The presentation unit 15 may generate support information for the user on the basis of the calculated user's motion intensity and display the generated support information on the display device 109, and may cause the above-described operation device to execute an operation of supporting the user on the basis of the support information.

The functions of the activity state analysis device 1 described above may not only be provided in one computer but may also be configured to be distributed to a plurality of computers that are communicatively connected to each other through a communication network.

### Activity State Analysis System

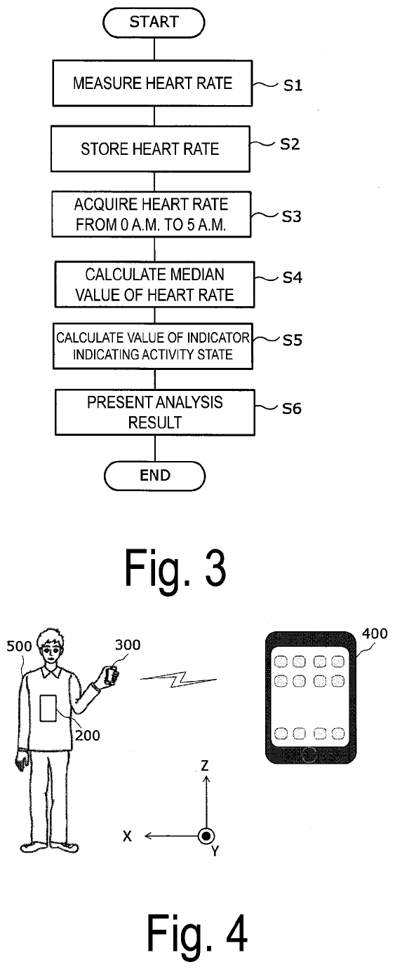

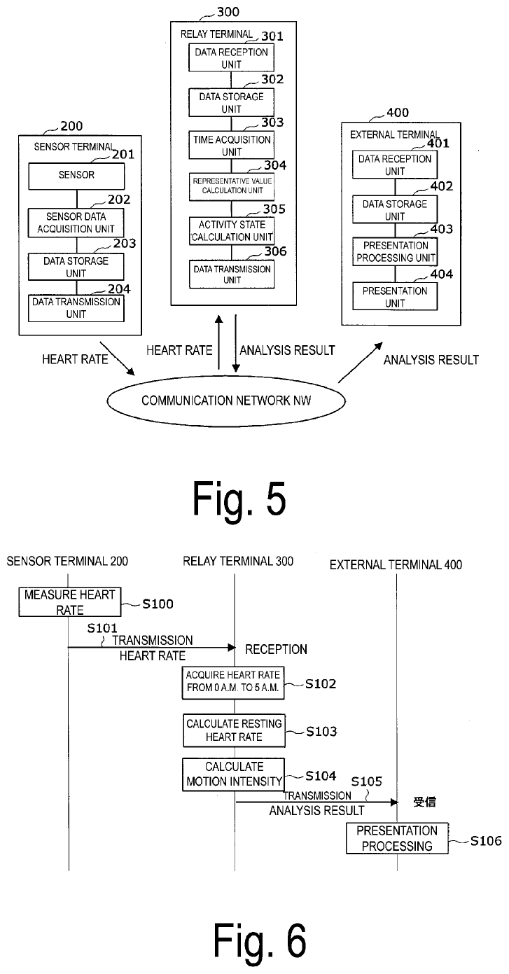

Next, an activity state analysis system in which the activity state analysis device 1 according to embodiments of the present disclosure is specifically configured will be described with reference to FIGS. 4 and 5.

For example, as illustrated in FIG. 4, the activity state analysis system includes a sensor terminal 200 worn by a user 500, a relay terminal 300, and an external terminal 400. All or any one of the sensor terminal 200, the relay terminal 300, and the external terminal 400 includes the functions of the activity state analysis device 1 such as the measurement unit 10, the representative value calculation unit 12, and the activity state calculation unit 14 described in FIG. 1. Note that a case where the relay terminal 300 includes the representative value calculation unit 12 and the activity state calculation unit 14 described in FIG. 1 will be described below.

### Functional Block of Sensor Terminal

The sensor terminal 200 includes a sensor 201, a sensor data acquisition unit 202, a data storage unit 203, and a data transmission unit 204. For example, the sensor terminal 200, which is placed on the trunk of the body of the user 500, measures a heart rate of the user. The sensor terminal 200 transmits the measured heart rate of the user 500 to the relay terminal 300 through the communication network NW.

The sensor 201 is implemented by a heart rate meter or the like. The sensor 201 detects and outputs a cardiac potential of the user. Data of the cardiac potential of the user is acquired by the sensor data acquisition unit 202. The sensor 201 corresponds to the sensor 106 described in FIG. 2.

The sensor data acquisition unit 202 acquires a heart rate from an electrocardiographic waveform based on the cardiac potential of the user 500 detected by the sensor 201. In more detail, the sensor data acquisition unit 202 performs noise removal and sampling processing on the electrocardiographic waveform, and obtains time-series data of a heart rate of a digital signal. The sensor data acquisition unit 202 corresponds to the measurement unit 10 described in FIG. 1. In the present embodiment, the sensor data acquisition unit 202 performs sampling of a heart rate, for example, at a sampling rate of one second.

The data storage unit 203 stores the electrocardiographic waveform based on the cardiac potential detected by the sensor 201 and time-series data of the heart rate of the digital signal obtained by being processed by the sensor data acquisition unit 202. The data storage unit 203 corresponds to the storage unit 13 (FIG. 1).

The data transmission unit 204 transmits the time-series data of the heart rate stored in the data storage unit 203 to the relay terminal 300 through the communication network NW. The data transmission unit 204 includes a communication circuit for performing wireless communication in compliance with wireless data communication standards such as LTE, 3G, a wireless local area network (LAN), or Bluetooth (registered trademark). The data transmission unit 204 corresponds to the transmission and reception unit 16 (FIG. 1).

### Functional Block of Relay Terminal

The relay terminal 300 includes a data reception unit 301, a data storage unit 302, a time acquisition unit 303, a representative value calculation unit 304, an activity state calculation unit 305, and a data transmission unit 306. The relay terminal 300 calculates a resting heart rate from the time-series data of the heart rate of the user 500 which is received from the sensor terminal 200 and calculates a motion intensity and the like using the calculated resting heart rate to analyze the activity state of the user 500. Further, the relay terminal 300 transmits analysis results to the external terminal 400.

The relay terminal 300 is implemented by a smart phone, a tablet, a laptop computer, or the like.

The data reception unit 301 receives time-series data of the heart rate from the sensor terminal 200 through the communication network NW. The data reception unit 301 corresponds to the transmission and reception unit 16 (FIG. 1).

The data storage unit 302 stores the heart rate of the user 500 which is received by the data reception unit 301 and information on a preset time period used by the representative value calculation unit 304. In the present embodiment, the data storage unit 302 stores information indicating a time period from 0 a.m. to 5 a.m. The data storage unit 302 stores the above-described Equations (1) and (2) for determining a motion intensity and a physiological cost index used by the activity state calculation unit 305. The data storage unit 302 corresponds to the storage unit 13 (FIG. 1).

The time acquisition unit 303 acquires a reference time used by the relay terminal 300. For example, the time acquisition unit 303 may acquire time information from an RTC or an NTP server including a crystal oscillator. Note that the time acquisition unit 303 may establish time synchronization between the sensor terminal 200 and the external terminal 400 using the time acquired by the time acquisition unit 303 as a reference time of the activity state analysis system. The time acquisition unit 303 corresponds to the time acquisition unit 11 illustrated in FIG. 1.

The representative value calculation unit 304 reads the information of the preset time period stored in the data storage unit 302 and acquires time-series data of the heart rate measured in the time period from the data storage unit 302 to calculate a median value (representative value) thereof. The calculated median value is stored in the data storage unit 302 as a resting heart rate. Note that the representative value calculation unit 304 may calculate an average value instead of the median value. The representative value calculation unit 304 corresponds to the representative value calculation unit 12 illustrated in FIG. 1.

The activity state calculation unit 305 substitutes the resting heart rate of the user 500 calculated by the representative value calculation unit 304 for Equation (1) described above to calculate a motion intensity. The calculated motion intensity is output as an analysis result of an activity state of the user 500. Note that the activity state calculation unit 305 may calculate a physiological cost index using Equation (2) described above. The activity state calculation unit 305 corresponds to the activity state calculation unit 14 illustrated in FIG. 1.

The data transmission unit 306 transmits the motion intensity and the physiological cost index calculated by the activity state calculation unit 305 to the external terminal 400 through the communication network NW as analysis results of the activity state. Note that the information transmitted to the external terminal 400 may include not only the motion intensity and the physiological cost index, but also the resting heart rate, the time-series data of the heart rate of the user 500, and the like. The data transmission unit 306 corresponds to the transmission and reception unit 16 (FIG. 1).

### Functional Block of External Terminal

The external terminal 400 includes a data reception unit 401, a data storage unit 402, a presentation processing unit 403, and a presentation unit 404. The external terminal 400 presents the analysis results of the activity state of the user 500 which are received from the relay terminal 300 through the communication network NW, and presents support information for the user 500 based on the analysis results.

Similar to the relay terminal 300, the external terminal 400 is implemented by a smart phone, a tablet, a laptop computer, or the like. The external terminal 400 includes a display device that displays the received analysis results, and an operation device (not illustrated) that outputs information for supporting the user 500 generated on the basis of the analysis results. Examples of the operating device included in the external terminal 400 include a display device, a sound output device, a light source, an actuator, heating equipment, and the like.

For example, a speaker or a musical instrument may be used as the sound output device. An LED or a light bulb may be used as the light source. An oscillator, a robot arm, or an electric treatment device may be used as the actuator. In addition, a heater, a Peltier element, or the like may be used as the heating equipment.

The data reception unit 401 receives the analysis results of the activity state from the relay terminal 300 through the communication network NW. The data reception unit 401 corresponds to the transmission and reception unit 16 (FIG. 1).

The data storage unit 402 stores the analysis results of the activity state received by the data reception unit 401. The data storage unit 402 corresponds to the storage unit 13 (FIG. 1).

The presentation processing unit 403 generates support information for the user 500 on the basis of the analysis results. The presentation processing unit 403 corresponds to the presentation unit 15 illustrated in FIG. 1.

The presentation unit 400 presents the analysis results and presents support information for the user 500 on the basis of an instruction by the presentation processing unit 403. In more detail, the analysis results and the support information may be displayed on the display device included in the external terminal 400 by using text information, a graph, or the like, and the support information may be output from a speaker, which is not illustrated in the drawing, included in the external terminal 400 by using an alert sound or the like. In addition, the presentation unit 400 can present the support information by a method perceptible by the user 500 such as vibration or light. The presentation unit 400 corresponds to the presentation unit 15 described in FIG. 1.

In this manner, the activity state analysis system according to embodiments of the present disclosure is configured such that the functions of the activity state analysis device 1 are distributed to the sensor terminal 200, the relay terminal 300, and the external terminal 400, and performs processing related to the measurement of a heart rate of the user 500, the calculation of a resting heart rate, and the presentation of the analysis results of the activity state in a distributed manner.

### Operation Sequence of Activity State Analysis System

Next, operations of the activity state analysis system having the above-described configuration will be described using the sequence diagram of FIG. 6.

As illustrated in FIG. 6, first, the sensor terminal 200 is worn by the user 500 and measures a heart rate (step S100). The sensor terminal 200 performs sampling of the heart rate, for example, at a sampling rate of one second to obtain a digital signal.

Next, the sensor terminal 200 transmits time-series data of the heart rate to the relay terminal 300 through the communication network NW (step S101). Note that the sensor terminal 200 may continuously measure the heart rate while the sensor terminal 200 is worn by the user 500, and may transmit the heart rate to the relay terminal 300 at fixed time intervals.

When the relay terminal 300 receives time-series data of the heart rate from the sensor terminal 200, the relay terminal 300 acquires time-series data of the heart rate measured in a set time period, that is, from 0 a.m. to 5 a.m. (step S102).

In more detail, when the relay terminal 300 receives the heart rate, the received heart rate is stored in the data storage unit 302. For example, the representative value calculation unit 304 may acquire the heart rate for five hours from the data storage unit 302 after 5 a.m. with reference to the current time acquired by the time acquisition unit 303.

Thereafter, the representative value calculation unit 304 of the relay terminal 300 calculates a median value (representative value) of the time-series data of the heart rate measured from 0 a.m. to 5 a.m. as a resting heart rate (step S103). Next, the activity state calculation unit 305 substitutes the resting heart rate of the user 500 calculated in step S103 for Equation (1) described above to obtain a motion intensity (step S104).

The relay terminal 300 transmits the motion intensity calculated in step S104 to the external terminal 400 through the communication network NW as an analysis result of the activity state of the user 500 (step S105). Note that the relay terminal 300 may transmit not only the motion intensity, but also the physiological cost index, the value of the resting heart rate, and the time-series data of the heart rate to the external terminal 400.

When the external terminal 400 receives the analysis result, the external terminal 400 executes presentation processing (step S106). Specifically, the external terminal 400 displays the analysis result on the display device. The external terminal 400 generates support information for the user 500 on the basis of the analysis result, and displays the support information on the display device or the like.

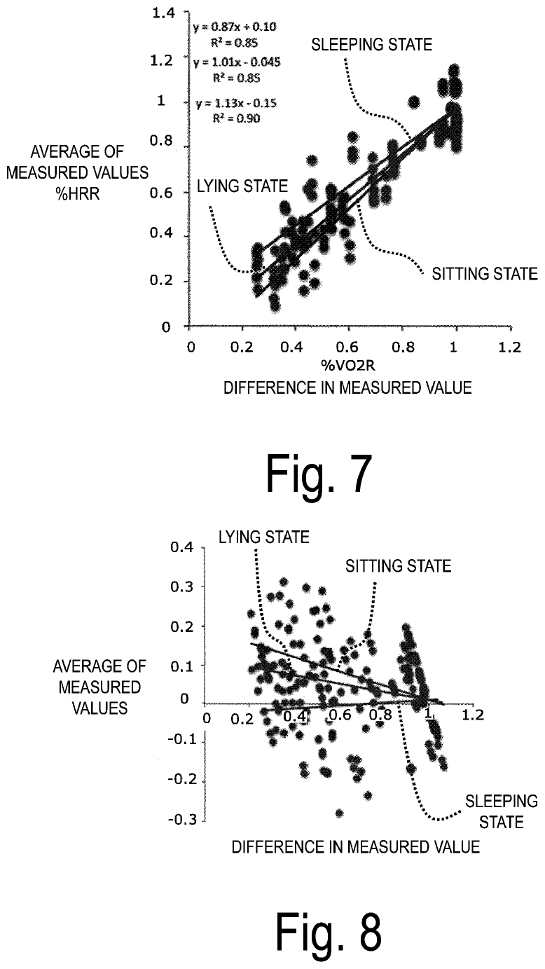

Next, effects of the activity state analysis device 1 according to the present embodiment will be described with reference to FIGS. 7 and 8. FIG. 7 is a diagram illustrating a relationship between motion intensity and oxygen intake for 12 adult men and women in their twenties. The vertical axis in FIG. 7 represents motion intensity (% HRR) and the horizontal axis represents oxygen intake (% VO₂R). When regression lines of measured values in a sitting position, a non-sleeping lying position, and a sleeping position are obtained, determination coefficients R² thereof are within the range of 0.85 to 0.9, and thus an extremely high correlation is obtained.

This indicates that, in the activity state analysis device 1 according to the present embodiment, it is possible to ascertain a user's metabolic function by measuring a heart rate of the user, for example, even when the user does not specially wear a mask or the like in a case where oxygen intake is measured.

Next, FIG. 8 illustrates results of Bland-Altman analysis performed on the measured values of motion intensity and oxygen intake in each of the sitting position, the non-sleeping lying position, and the sleeping position illustrated in FIG. 7. In the Bland-Altman analysis, a regression line is closer to the horizontal axis in a case where there is no error due to a bias, and a regression line is away from the horizontal axis and is rotated when there is an influence of an error.

As illustrated in FIG. 8, an error is not observed in only the measured values in the sleeping position among the results in the cases of the sitting position, the non-sleeping lying position, and the sleeping position, and thus it can be understood that highly reliable results are obtained. A value indicating an activity state based on a resting heart rate calculated in a lying position or a sitting position in a situation where the user is conscious includes a larger error. On the other hand, an activity state based on a resting heart rate calculated from a heart rate in the sleeping position when the user is not conscious has little influence of an error. The activity state analysis device 1 according to the present embodiment can obtain a stable resting heart rate using a simpler configuration even in a situation where the user is sleeping in an unconscious state. For this reason, it is possible to ascertain a highly reliable activity state.

As described above, the activity state analysis device 1 according to the first embodiment calculates a representative value of time-series data of the user's heart rate measured in a set time period as a resting heart rate. Thus, it is possible to obtain a highly reliable indicator indicating an activity state of the user on the basis of the value of a stable resting heart rate.

Further, in the above-described embodiment, a resting heart rate is obtained on the basis of a user's heart rate, which is the number of beats in which the heart sends blood to the whole body, but a resting heart rate can be calculated using a pulse rate generated in the arteries in the whole body instead of using the heart rate. Thus, it is possible to obtain an indicator indicating a highly reliable activity state of the user on the basis of biological information that can be measured more easily.

### Second Embodiment

Next, a second embodiment of the present disclosure will be described. Note that, in the following description, the same components as those in the above-described first embodiment will be denoted by the same reference numerals and signs, and descriptions thereof will be omitted.

In the first embodiment, a case where the representative value calculation unit 12 calculates a representative value of time-series data of a user's heart rate measured in a set time period as a resting heart rate has been described. On the other hand, in the second embodiment, an activity state analysis device 1A further includes a lying position estimation unit 17 and estimates a lying position of a user. Then, a representative value calculation unit 12 calculates a representative value of a heart rate measured at a plurality of points in time at which a lying position is estimated as a resting heart rate. Hereinafter, different configurations from those in the first embodiment will be mainly described.

In addition, a case where a heart rate is adopted as an example of biological information used for analysis by the activity state analysis device IA will be described below, but a resting heart rate may be obtained using a pulse rate instead of a heart rate as in the first embodiment.

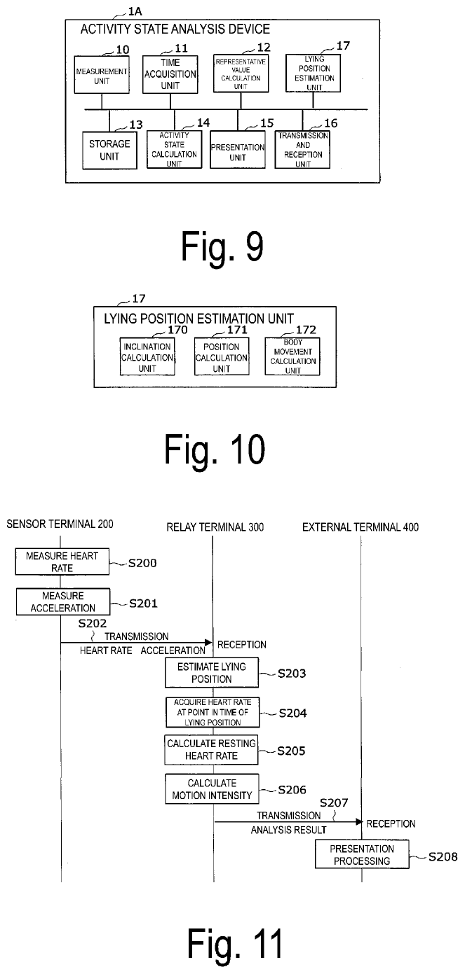

As illustrated in FIG. 9, the activity state analysis device 1A includes the lying position estimation unit 17. Further, in the present embodiment, a sensor 106 worn by the user includes a three-axis acceleration sensor.

The lying position estimation unit 17 estimates the lying position of the user on the basis of acceleration data of the user which is detected by the three-axis acceleration sensor (the sensor 106). As illustrated in FIG. 10, the lying position estimation unit 17 includes an inclination calculation unit 170, a position calculation unit 171, and a body movement calculation unit 172.

The inclination calculation unit 170 calculates an inclination of the sensor 106 from three-axis acceleration data.

The position calculation unit 171 calculates the position of the user from the inclination calculated by the inclination calculation unit 170. The value calculated by the position calculation unit 171 is stored in the storage unit 13 in association with time information in which acceleration data is detected. Information of the position of the user calculated by the position calculation unit 171 is used for the estimation of the user's lying position by the lying position estimation unit 17.

The body movement calculation unit 172 calculates the magnitude of a body movement of the user from three-axis acceleration data.

Here, the inclination calculation unit 170 calculates θ and φ according to the following equation as an inclination of the sensor 106 with respect to a gravitational acceleration from the three-axis acceleration data. Note that the inclination calculation unit 170 uses the three-axis acceleration data sampled, for example, at a sampling rate of 25 Hz.

Further, θ (−90≤θ<270) is an inclination of the Z axis of an acceleration sensor with respect to a vertical direction, φ (−90≤φ<270) is an inclination of the X axis of the acceleration sensor with respect to the vertical direction, and the units thereof are degrees. Note that the X, Y, and Z axes represent axes of the acceleration sensor which are orthogonal to each other.

$$\begin{matrix}Equation ( 3 ) & \\ \begin{matrix}θ = \frac{180}{π} {cos}^{- 1} ( \frac{{A}_{z}}{\sqrt{{A}_{x}^{2} + {A}_{y}^{2} + {A}_{z}^{2}}} ) + 90 & ( {A}_{y} ≧ 0 Case of ) \\ θ = - \frac{180}{π} {cos}^{- 1} ( \frac{{A}_{z}}{\sqrt{{A}_{x}^{2} + {A}_{y}^{2} + {A}_{z}^{2}}} ) + 90 & ( {A}_{y} < 0 Case of )\end{matrix} & ( 3 ) \\ Equation ( 4 ) & \\ \begin{matrix}ϕ = \frac{180}{π} {cos}^{- 1} ( \frac{{A}_{x}}{\sqrt{{A}_{x}^{2} + {A}_{y}^{2} + {A}_{z}^{2}}} ) + 90 & ( {A}_{y} ≧ 0 Case of ) \\ ϕ = - \frac{180}{π} {cos}^{- 1} ( \frac{{A}_{x}}{\sqrt{{A}_{x}^{2} + {A}_{y}^{2} + {A}_{z}^{2}}} ) + 90 & ( {A}_{y} < 0 Case of )\end{matrix} & ( 4 )\end{matrix}$$

Ax, Ay, and Az are output values of the acceleration sensor, and the units thereof are a gravitational acceleration G (1.0 G≈9.8 m/s2). In Equations (3) and (4), a ratio of a measured value of a single axis with respect to the magnitude (norm) of a synthetic vector of an output value of the acceleration sensor is obtained, and an inverse function of a cosine is obtained, thereby calculating a value having an angle dimension.

Regarding Ax, Ay, and Az in Equations (3) and (4), an output value of the acceleration sensor may be substituted as it is, or a value obtained by applying a low-pass filter (for example, an FIR filter or a moving average filter) for smoothing may be used.

The position calculation unit 171 calculates a position by comparing the values of θ and φ calculated in Equations (3) and (4) with threshold values. For example, an inclination of the sensor terminal 200 including the acceleration sensor as illustrated in FIG. 4 reflects an inclination of the upper body of the user 500 wearing the sensor terminal 200, and thus it is possible to calculate the position of the user 500 from the inclination of the sensor terminal 200. For example, positions according to cases are calculated using the following calculation method. Note that, regarding the three axes of the acceleration sensor included in the sensor 201, the X axis is provided in parallel with the right-left direction of the body, the Y axis is provided in parallel with the front-back direction of the body, and the Z axis is provided in parallel with the up-down direction of the body, for example, as illustrated in FIG. 5.

(i) Standing position (upright): Case of 30≤θ<140.

(ii) Standing position (handstand): Case of θ<−40 or 220<0.

(iii) Lying position (left half of the body is positioned upward): Case of (φ≤−50 or 230<φ) and (−40≤θ<30), or case of (φ≤−50 or 230<φ) and (140<0<220).

(iv) Lying position (right half of the body is positioned upward): Case of (50<φ<130) and (−40≤θ<30), or case of (50<φ<130) and (140<0<220).

(v) Lying position (lying on back): Case of (130≤φ≤230) and (−40≤θ<30), or case of (130≤φ≤230) and (140<θ<220).

(vi) Lying position (lying face-up): Case of (−50≤φ≤50) and −40≤φ<30), or case of (−50≤φ≤50) and (140<θ<220).

Definitions of (i) to (vi) in the calculations described above need to be set (stored) in the position calculation unit 171 as a table of θ and φ as shown in Table 1 below.

TABLE 1

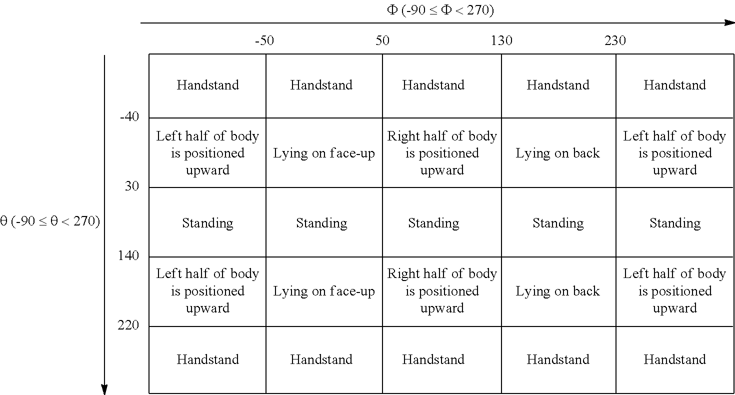

In this manner, the position of the user calculated by the position calculation unit 171 is stored in the storage unit 13 in association with the time at which acceleration data is detected.

The lying position estimation unit 17 identifies a point in time when the user is in the lying position among the user's positions calculated by the position calculation unit 171.

The representative value calculation unit 12 acquires a plurality of heart rate measurements measured at a plurality of points in time when the user is estimated to be in a lying position by the lying position estimation unit 17 from the storage unit 13 to calculate a median value (representative value) thereof. The calculated median value is stored in the storage unit 13 as a resting heart rate in the user's resting state.

Here, in daily life, the user's lying position is not necessarily limited to a case where the user is sleeping. However, generally considering that bedtime hours are approximately 6 to 8 hours, most of a period of a lying position may be considered to be bedtime hours even when the lying period is 12 hours. For this reason, the representative value calculation unit 12 calculates a representative value of a plurality of heart rate measurements at a plurality of points in time when the user is in a lying position.

In addition, the representative value calculation unit 12 takes a median value of the plurality of heart rate measurements in a period in which the user is in a lying position, and thus there is little influence from exceptional lying position in a period of time other than a period of time in which the user is sleeping.

### Operation Sequence of Activity State Analysis System

Next, operations in a case where the functions of the activity state analysis device 1A according to the present embodiment are implemented by the activity state analysis system including the sensor terminal 200, the relay terminal 300, and the external terminal 400 illustrated in FIG. 4 will be described with reference to the sequence diagram illustrated in FIG. 11. Note that the functional blocks of the sensor terminal 200, the relay terminal 300, and the external terminal 400 are the same as the configuration illustrated in FIG. 4. In addition, it is assumed that the relay terminal 300 includes the lying position estimation unit 17, the representative value calculation unit 12, and the activity state calculation unit 14. Further, the sensor 201 of the sensor terminal 200 includes a heart rate meter and a three-axis acceleration sensor.

First, the sensor terminal 200, which is worn by the user 500, measures a heart rate of the user 500 (step S200). In more detail, the sensor terminal 200 measures a cardiac potential of the user 500 using a heart rate meter (the sensor 201). The sensor data acquisition unit 202 acquires the cardiac potential from the sensor 201 and calculates a heart rate of digital data from an electrocardiographic waveform based on the cardiac potential. The acquired cardiac potential and heart rate are stored in the data storage unit 203.

Next, the sensor terminal 200 measures accelerations of three axes of the user 500 (step S201). Thereafter, the sensor terminal 200 transmits time-series data of the measured heart rates and accelerations to the relay terminal 300 through the communication network NW (step S202). For example, the sensor terminal 200 may transmit data of the heart rates and the accelerations at fixed time intervals. In addition, the sensor terminal 200 may transmit the heart rates and the accelerations separately.

When the relay terminal 300 receives the time-series data of the heart rates and the accelerations of the user 500 from the sensor terminal 200, the relay terminal 300 first estimates the lying position of the user 500 on the basis of the received three-axis acceleration data by the lying position estimation unit 17 (step S203).

In more detail, the inclination calculation unit 170 obtains an inclination of the acceleration sensor (the sensor 201) according to Equations (3) and (4) described above on the basis of the three-axis acceleration data. Thereafter, the position calculation unit 171 calculates the position of the user 500 with reference to Table 1 described above on the basis of the inclination calculated by the inclination calculation unit 170. Then, the lying position estimation unit 17 identifies a point in time of the lying position from the calculated position of the user 500. Additionally, the body movement calculation unit 172 calculates the magnitude of the body movement of the user 500 from the three-axis acceleration data. The lying position of the user 500 estimated by the lying position estimation unit 17 is stored in the data storage unit 302 in association with the time at which the corresponding acceleration data is detected.

Thereafter, the representative value calculation unit 304 of the relay terminal 300 acquires heart rates measured in a period in which the user 500 is in a lying position from the data storage unit 302 (step S204).

Next, the representative value calculation unit 304 calculates a median value of the plurality of heart rate measurements acquired in step S204 as a resting heart rate (step S205). Note that the representative value calculation unit 304 may calculate an average value as a resting heart rate instead of the median value.

Thereafter, the activity state calculation unit 305 calculates a motion intensity according to Equation (1) described above on the basis of the resting heart rate calculated in step S205 (step S206). Note that the activity state calculation unit 305 may calculate a physiological cost index according to Equation (2) described above using the resting heart rate.

Next, the relay terminal 300 transmits the calculated motion intensity to the external terminal 400 through the communication network NW as an analysis result of an activity state (step S207). Thereafter, when the external terminal 400 receives the analysis result, the external terminal 400 performs presentation processing (step S208). In more detail, the external terminal 400 performs the presentation processing on the basis of the analysis result (step S205), displays the analysis result on the display device, and generates and outputs supporting information for the user 500.

As described above, according to the activity state analysis device 1 of the second embodiment, a representative value of a plurality of heart rate measurements at a plurality of points in time when a user is estimated to be in a lying position is calculated as a resting heart rate, and thus it is possible to obtain a more stable resting heart rate. Thus, it is possible to obtain a more reliable indicator indicating an activity state of the user.

### Third Embodiment

Next, a third embodiment of the present disclosure will be described. Note that, in the following description, the same components as those in the above-described first and second embodiments will be denoted by the same reference numerals and signs, and descriptions thereof will be omitted.

In the first embodiment, a representative value of time-series data of heart rates in a set time period is calculated as a resting heart rate. Further, in the second embodiment, a lying position of a user is estimated, and a representative value of a plurality of heart rate measurements at a plurality of points in time when the user is in a lying position is calculated as a resting heart rate. On the other hand, in the third embodiment, the representative value calculation unit 12 calculates a representative value of a plurality of heart rate measurements at a plurality of points in time when a user is estimated to be in a lying position in a set time period as a resting heart rate. Hereinafter, different configurations from those in the first and second embodiments will be mainly described.

Note that a case where a heart rate is adopted as an example of biological information used for analysis by the activity state analysis device 1A will be described below, but a resting heart rate may be obtained using a pulse rate instead of the heart rate, similar to the first and second embodiments.

A configuration of the activity state analysis device IA according to the present embodiment is the same as the functional configuration illustrated in FIG. 9.

The representative value calculation unit 12 acquires a plurality of heart rate measurements measured when a user is resting from the storage unit 13 to calculate a representative value. In more detail, the representative value calculation unit 12 calculates a median value (representative value) of a plurality of heart rate measurements at a plurality of points in time when the lying position estimation unit 17 estimates that the user is in a lying position, among heart rates measured in a time period from 0 a.m. to 5 a.m., as a resting heart rate.

### Operation Sequence of Activity State Analysis System

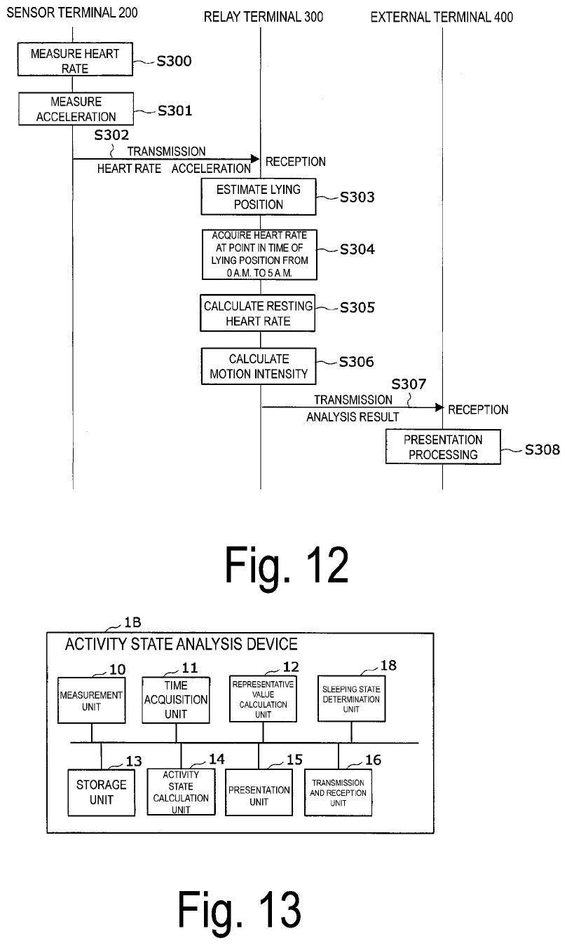

Next, operations in a case where the functions of the activity state analysis device 1A according to the present embodiment are realized by the activity state analysis system including the sensor terminal 200, the relay terminal 300, and the external terminal 400 illustrated in FIG. 4 will be described with reference to the sequence diagram illustrated in FIG. 12. Note that the functional blocks of the sensor terminal 200, the relay terminal 300, and the external terminal 400 are the same as the configuration illustrated in FIG. 4. In addition, it is assumed that the relay terminal 300 includes the lying position estimation unit 17 and the representative value calculation unit 12. Further, the sensor 201 of the sensor terminal 200 includes a heart rate meter and a three-axis acceleration sensor.

First, the sensor terminal 200, which is worn by the user 500, measures a heart rate of the user 500 (step S300). In more detail, the sensor terminal 200 measures a cardiac potential of the user 500 by using a heart rate meter (the sensor 201). The sensor data acquisition unit 202 acquires the cardiac potential from the sensor 201 and calculates a heart rate of digital data from an electrocardiographic waveform based on the cardiac potential. The acquired cardiac potential and heart rate are stored in the data storage unit 203.

Next, the sensor terminal 200 measures accelerations of three axes of the user 500 (step S301). Thereafter, the sensor terminal 200 transmits time-series data of the measured heart rates and accelerations to the relay terminal 300 through the communication network NW (step S302). For example, the sensor terminal 200 may transmit data of the heart rates and the accelerations at fixed time intervals. In addition, the sensor terminal 200 may transmit the heart rates and the accelerations separately.

When the relay terminal 300 receives the time-series data of the heart rates and the accelerations of the user 500 from the sensor terminal 200, the relay terminal 300 first estimates the lying position of the user 500 on the basis of the received three-axis acceleration data by the lying position estimation unit 17 (step S303).

In more detail, the inclination calculation unit 170 obtains an inclination of the acceleration sensor (the sensor 201) according to Equations (3) and (4) described above on the basis of the three-axis acceleration data. Thereafter, the position calculation unit 171 calculates the position of the user 500 with reference to Table 1 described above on the basis of the inclination calculated by the inclination calculation unit 170. The lying position estimation unit 17 identifies a point in time at which the user 500 is in a lying position among the calculated positions. Additionally, the body movement calculation unit 172 calculates the magnitude of the body movement of the user 500 from the three-axis acceleration data. The lying state of the user 500 estimated by the lying position estimation unit 17 is stored in the data storage unit 302 in association with the time at which the corresponding acceleration data is detected.

Thereafter, the representative value calculation unit 304 of the relay terminal 300 reads information on a set time period stored in the data storage unit 302, that is, a period from 0 a.m. to 5 a.m. The representative value calculation unit 304 acquires a plurality of heart rate measurements of the user 500 which are measured at a plurality of points in time when the user 500 is in a lying position in the time period from 0 a.m. to 5 a.m. from the data storage unit 302 (step S304).

Next, the representative value calculation unit 304 calculates a median value of the plurality of heart rate measurements acquired in step S304 as a resting heart rate (step S305).

Note that the representative value calculation unit 304 may calculate an average value as a resting heart rate instead of the median value.

Thereafter, the activity state calculation unit 305 calculates a motion intensity according to Equation (1) described above on the basis of the resting heart rate calculated in step S305 (step S306). Note that the activity state calculation unit 305 may calculate a physiological cost index according to Equation (2) described above using the resting heart rate.

Next, the relay terminal 300 transmits the calculated motion intensity to the external terminal 400 through the communication network NW as an analysis result of an activity state (step S307). Thereafter, when the external terminal 400 receives the analysis result, the external terminal 400 performs presentation processing (step S308). In more detail, the external terminal 400 performs the presentation processing on the basis of the analysis result (step S305), displays the analysis result on the display device, and generates and outputs supporting information for the user 500.

As described above, according to the activity state analysis device 1 of the third embodiment, a representative value of a plurality of heart rate measurements at a plurality of points in time when a user is in a lying position from 0 a.m. to 5 a.m. is calculated as a resting heart rate, and thus it is possible to obtain a more stable resting heart rate. Thus, it is possible to obtain a more reliable indicator indicating an activity state of the user.

### Fourth Embodiment

Next, a fourth embodiment of the present disclosure will be described. Note that, in the following description, the same components as those in the above-described first to third embodiments will be denoted by the same reference numerals and signs, and descriptions thereof will be omitted.

In the second embodiment, the lying position estimation unit 17 estimates a lying position of a user. Then, the representative value calculation unit 12 calculates a representative value of a plurality of heart rate measurements at a plurality of points in time when the user is in a lying position as a resting heart rate. On the other hand, in the fourth embodiment, an activity state analysis device 1B, further including a sleeping state determination unit 18, determines a state in which a user is sleeping. Then, the representative value calculation unit 12 calculates, as a resting heart rate, a representative value of a plurality of heart rate measurements at a plurality of points in time when it is determined that the user is in a sleeping state. Hereinafter, different configurations from those in the first to third embodiments will be mainly described.

FIG. 13 is a block diagram illustrating a functional configuration of the activity state analysis device 1B according to the present embodiment. The activity state analysis device 1B includes the sleeping state determination unit 18. The other functional configurations of the activity state analysis device 1B are the same as the functional configurations described in the first to third embodiments.

The sleeping state determination unit 18 determines a sleeping state from a heart rate, an acceleration, or the like of the user which is measured by a heart rate meter or an acceleration sensor (not illustrated). For example, the sleeping state determination unit 18 can use a technique disclosed in Non Patent Literature 4. In more detail, it is possible to determine a sleeping state of the user by combining determination results of a sleeping state using Cole's algorithm and ESS algorithm by machine learning such as support vector machine (SVM), k-nearest neighbor algorithm, or random forest. Note that Cole's algorithm is an algorithm for calculating a body movement from a measured acceleration of a user and determining a sleeping state on the basis of the number of body movements. In addition, ESS algorithm is an algorithm for determining a sleeping state on the basis of calculated body motion intervals.

In addition, the sleeping state determination unit 18 may analyze features of a body movement when the user is sleeping from a pattern of the measured acceleration of the user, using a wearable wristband type sensor, disclosed in Non Patent Literature 5, which measures a heart rate and an acceleration, and may determine a sleeping state.

Additionally, the sleeping state determination unit 18 may determine a sleeping state of a user on the basis of three types of vibrations, that is, breathing, pulse, and body movement of the user by using a mattress type sleep scan disclosed in Non Patent Literature 6.

The representative value calculation unit 12 acquires a plurality of heart rate measurements at a plurality of points in time when the user is estimated to be in a sleeping state from the storage unit 13 to calculate a representative value thereof. In more detail, the representative value calculation unit 12 acquires a plurality of heart rate measurements at a plurality of points in time when the sleeping state determination unit 18 determines that the user is in a sleeping state from the storage unit 13 to calculate a median value (representative value) thereof. The calculated median value of the plurality of heart rate measurements is stored in the storage unit 13 as a resting heart rate.

### Operation Sequence of Activity State Analysis System

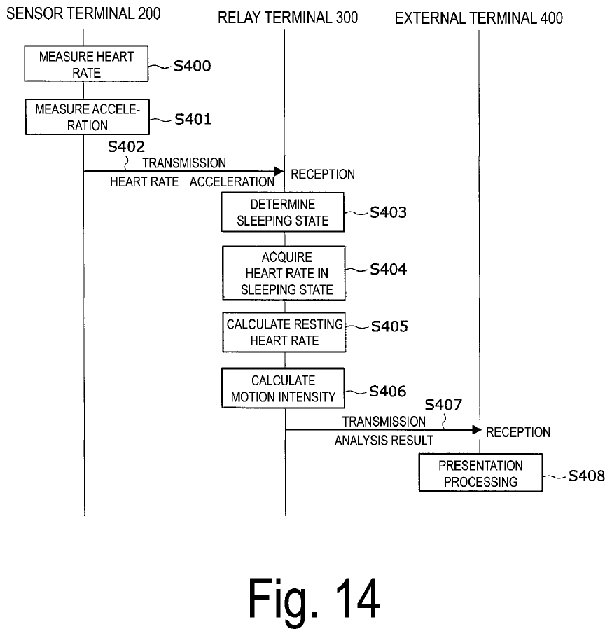

Next, operations in a case where the functions of the activity state analysis device 18 according to the present embodiment are implemented by the activity state analysis system including the sensor terminal 200, the relay terminal 300, and the external terminal 400 illustrated in FIG. 4 will be described with reference to the sequence diagram illustrated in FIG. 14. Note that the functional blocks of the sensor terminal 200, the relay terminal 300, and the external terminal 400 are the same as the configuration illustrated in FIG. 4. In addition, it is assumed that the relay terminal 300 includes the sleeping state determination unit 18, the representative value calculation unit 12, and the activity state calculation unit 14. Further, the sensor 201 of the sensor terminal 200 includes a heart rate meter and a single-axis or three-axis acceleration sensor.

First, the sensor terminal 200, which is worn by the user 500, measures a heart rate of the user 500 (step S400). In more detail, the sensor terminal 200 measures a cardiac potential of the user 500 using a heart rate meter (the sensor 201). The sensor data acquisition unit 202 acquires the cardiac potential from the sensor 201 and calculates a heart rate of digital data from an electrocardiographic waveform based on the cardiac potential. The acquired cardiac potential and heart rate are stored in the data storage unit 203.

Next, the sensor terminal 200 measures accelerations of the user 500 (step S401). Thereafter, the sensor terminal 200 transmits time-series data of the measured heart rates and accelerations to the relay terminal 300 through the communication network NW (step S402). For example, the sensor terminal 200 may transmit data of the heart rates and the accelerations at fixed time intervals. In addition, the sensor terminal 200 may transmit the heart rates and the accelerations separately.

When the relay terminal 300 receives the time-series data of the heart rates and the accelerations of the user 500 from the sensor terminal 200, the relay terminal 300 first determines a sleeping state of the user 500 on the basis of the received acceleration data by the sleeping state determination unit 18 (step S403).

Thereafter, the representative value calculation unit 304 (the representative value calculation unit 12) of the relay terminal 300 acquires a plurality of heart rate measurements at a plurality of points in time when the user 500 is in a sleeping state from the data storage unit 302 (step S404).

Next, the representative value calculation unit 304 calculates a median value of the plurality of heart rate measurements acquired in step S404 as a resting heart rate (step S405). Note that the representative value calculation unit 304 may calculate an average value as a resting heart rate instead of the median value.

Thereafter, the activity state calculation unit 305 calculates a motion intensity according to Equation (1) described above on the basis of the resting heart rate calculated in step S405 (step S406). Note that the activity state calculation unit 305 may calculate a physiological cost index according to Equation (2) described above using the resting heart rate.

Next, the relay terminal 300 transmits the calculated motion intensity to the external terminal 400 through the communication network NW as an analysis result of an activity state (step S407). Thereafter, when the external terminal 400 receives the analysis result, the external terminal 400 performs presentation processing (step S408). In more detail, the external terminal 400 performs the presentation processing on the basis of the analysis result, displays the analysis result on the display device, and generates and outputs supporting information for the user 500.

As described above, according to the activity state analysis device 1B according to the fourth embodiment, a representative value of a plurality of heart rate measurements at a plurality of points in time when the user is in a sleeping state is calculated as a resting heart rate, and thus it is possible to obtain a more stable resting heart rate. Thus, it is possible to obtain a highly reliable indicator indicating an activity state of the user.

Although the activity state analysis device, the activity state analysis method, and the activity state analysis system of embodiments of the present disclosure have been described above, the present disclosure is not limited to the embodiments described above, and various modifications conceivable by a person skilled in the art can be made within the scope of the invention recited in the claims.

For example, in the embodiments described above, a case where a motion intensity and a physiological cost index are calculated using a resting heart rate has been described. However, the present disclosure is not limited thereto as long as an indicator for analyzing an activity state of a user using a resting heart rate is used.

Further, in the embodiments described above, in the specific examples, a case where the relay terminal 300 includes the representative value calculation unit 12 has been described. However, the functions of the representative value calculation unit 12 may be implemented distributedly by the sensor terminal 200, the relay terminal 300, and the external terminal 400.

For example, the sensor terminal 200 including a first analysis unit, the relay terminal 300 including a second analysis unit, and the external terminal 400 including a third analysis unit may calculate a representative value of time-series data of biological information including a heart rate or a pulse rate of a user which are measured in a set time period as a resting heart rate in cooperation with each other.

In addition, the sensor terminal 200 including the first analysis unit, the relay terminal 300 including the second analysis unit, and the external terminal 400 including the third analysis unit may calculate the following value as a resting heart rate in cooperation with each other. The following value refers to a representative value of a plurality of heart rate measurements or pulse rates measurements at a plurality of points in time when a user is estimated to be in a lying position by the lying position estimation unit 17.

Further, the sensor terminal 200 including the first analysis unit, the relay terminal 300 including the second analysis unit, and the external terminal 400 including the third analysis unit may calculate the following value as a resting heart rate in cooperation with each other. The following value refers to a representative value of a plurality of heart rate measurements or pulse rates measurements at a plurality of points in time when the sleeping state determination unit 18 determines that a user is in a sleeping state.

Similarly, the function of the activity state calculation unit 14 may be implemented distributedly by the sensor terminal 200, the relay terminal 300, and the external terminal 400.

### REFERENCE SIGNS LIST

1 Activity state analysis device

10 Measurement unit

11 Time acquisition unit

12, 304 Representative value calculation unit

13 Storage unit

14, 305 Activity state calculation unit

15, 404 Presentation unit

16 Transmission and reception unit

101 Bus

102 Arithmetic device

103 CPU

104 Main memory device

105 Communication interface

106, 201 Sensor

107 External storage device

108 Timepiece

109 Display device

200 Sensor terminal

300 Relay terminal

400 External terminal

202 Sensor data acquisition unit

203, 302, 402 Data Storage unit

204, 306 Data Transmission unit

301, 401 Data Reception unit

403 Presentation processing unit

1-8. (canceled)

9. An activity state analysis device comprising:
a measurer configured to measure a piece of biological information including a heart rate or a pulse rate of a user;
a storage configured to store a plurality of the pieces of biological information measured in time series;
a representative value calculator configured to acquire, from the storage, the plurality of the pieces of biological information measured when the user is in a resting state and calculate a representative value; and
an activity state calculator configured to calculate an activity state of the user by using the representative value calculated by the representative value calculator.

10. The activity state analysis device according to claim 9, wherein the plurality of the pieces of biological information measured when the user is in the resting state comprise the plurality of the pieces of biological information measured in a set time period.

11. The activity state analysis device according to claim 9, wherein the plurality of the pieces of biological information measured when the user is in the resting state comprise the plurality of the pieces of biological information measured at a plurality of points in time when the user is estimated to be in a lying position based on an acceleration of the user.

12. The activity state analysis device according to claim 9, wherein the plurality of the pieces of biological information measured when the user is in the resting state comprise the plurality of the pieces of biological information measured at a plurality of points in time when the user is estimated to be in a sleeping state.

13. An activity state analysis method comprising:
a measurement step of measuring a piece of biological information including a heart rate or a pulse rate of a user;
a storage step of storing a plurality of the pieces of biological information measured in time series in a storage;
a representative value calculation step of acquiring, from the storage, the plurality of the pieces of biological information measured when the user is in a resting state and calculating a representative value; and
an activity state calculation step of calculating an activity state of the user by using the representative value calculated in the representative value calculation step.

14. The activity state analysis method according to claim 13, wherein the plurality of the pieces of biological information measured when the user is in the resting state comprise the plurality of the pieces of biological information measured in a set time period.

15. The activity state analysis method according to claim 13, wherein the plurality of the pieces of biological information measured when the user is in the resting state comprise the plurality of the pieces of biological information measured at a plurality of points in time when the user is estimated to be in a lying position based on an acceleration of the user.

16. The activity state analysis method according to claim 13, wherein the plurality of the pieces of biological information measured when the user is in the resting state comprise the plurality of the pieces of biological information measured at a plurality of points in time when the user is estimated to be in a sleeping state.

17. An activity state analysis system comprising:
a sensor terminal configured to output a piece of biological information including a heart rate or a pulse rate of a user which are measured by a sensor;
a relay terminal configured to receive the piece of biological information output from the sensor terminal and output the piece of biological information; and
an external terminal configured to receive the piece of biological information output from the sensor terminal or the relay terminal and display the piece of biological information on a display device,
wherein at least one of the sensor terminal, the relay terminal, or the external terminal includes:
a storage configured to store a plurality of the pieces of biological information measured in time series;
a representative value calculator configured to acquire, from the storage, the plurality of the pieces of biological information measured when the user is in a resting state and calculate a representative value; and
an activity state calculator configured to calculate an activity state of the user using the representative value calculated by the representative value calculator.

18. The activity state analysis system according to claim 17, wherein:
the sensor terminal includes a first analyzer;
the relay terminal includes a second analyzer;
the external terminal includes a third analyzer; and
the first analyzer, the second analyzer, and the third analyzer are configured to cause, in cooperation with each other, the representative value calculator and the activity state calculator to be implemented.

19. The activity state analysis system according to claim 17, wherein the plurality of the pieces of biological information measured when the user is in the resting state comprise the plurality of the pieces of biological information measured in a set time period.

20. The activity state analysis system according to claim 17, wherein the plurality of the pieces of biological information measured when the user is in the resting state comprise the plurality of the pieces of biological information measured at a plurality of points in time when the user is estimated to be in a lying position based on an acceleration of the user.

21. The activity state analysis system according to claim 17, wherein the plurality of the pieces of biological information measured when the user is in the resting state comprise the plurality of the pieces of biological information measured at a plurality of points in time when the user is estimated to be in a sleeping state.

22. The activity state analysis system according to claim 17, wherein the representative value is a median value.

23. The activity state analysis system according to claim 17, wherein the representative value is an average value.
